(19) United States
(12) Patent Application Publication
(10) Pub. No.: US 2025/0090717 A1
(43) Pub. Date: Mar. 20, 2025

(54) THERAPEUTIC USE OF FIBROBLASTS FOR ADHESIVE BANDAGE WOUND HEALING

(71) Applicant: FIBROBIOLOGICS, INC., Houston, TX (US)

(72) Inventors: Pete O'HEERON, Houston, TX (US); Hamid KHOJA, Houston, TX (US)

(73) Assignee: FIBROBIOLOGICS, INC., Houston, TX (US)

(21) Appl. No.: 18/808,529

(22) Filed: Aug. 19, 2024

Related U.S. Application Data
(60) Provisional application No. 63/583,171, filed on Sep. 15, 2023.

(51) Int. Cl. A61L 15/44; A61L 15/40; A61L 26/00

(52) U.S. Cl. CPC A61L 15/44; A61L 15/40; A61L 26/0057; A61L 26/0066; A61L 2300/412; A61L 2300/64

(57) ABSTRACT

Embodiments of the disclosure include methods, compositions, and systems for wound care utilizing a wound coverage substrate utilizing dehydrated or lyophilized non-live fibroblasts and/or dehydrated or lyophilized fibroblast-derived materials. In particular embodiments, the wound coverage substrate comprises the dehydrated or lyophilized non-live fibroblasts and/or dehydrated or lyophilized fibroblast-derived materials and upon delivery to the wound, the moisture of the wound re-hydrates the dehydrated or lyophilized non-live fibroblasts and/or dehydrated or lyophilized fibroblast-derived materials.

### CROSS-REFERENCE TO RELATED APPLICATION

This application claims priority to U.S. Provisional Patent Application Ser. No. 63/583,171, filed Sep. 15, 2023, which is incorporated by reference herein in its entirety.

### BACKGROUND OF THE DISCLOSURE

### I. Technical Field

Embodiments of the disclosure concern at least the fields of cell biology, molecular biology, immunology, and medicine.

### II. Background

Wound healing is a complex process involving a complex orchestrated cellular and molecular interactions [1]. Traditional bandages lack the ability to actively contribute to the healing process. While they provide a barrier to external contaminants, they do not provide a sufficiently moist environment for wound healing or additional therapeutic benefits [2]. The current invention aims to address this limitation by incorporating fibroblasts and/or fibroblast derived materials such as, exosomes, lysates, membranes, and/or apoptotic bodies onto a bandage, thereby creating an advanced wound care solution. Growth factors, cytokines and/or chemokines can also be included.

### BRIEF SUMMARY

Embodiments of the disclosure encompass methods, compositions, and systems for wound care of any kind. In specific embodiments, the methods, compositions, and systems comprise fibroblasts, including non-live fibroblasts, and/or fibroblast-derived materials from single fibroblast cell culture, or spheroid fibroblast cell culture, for treating one or more wounds of any kind. The wounds may be on an individual that is a mammal of any kind, including a primate of any kind. In specific embodiments, the individual is a human, dog, cat, cow, horse, pig, and so forth.

In particular embodiments, the non-live fibroblasts, and/or fibroblast-derived materials from single fibroblast cell culture, or spheroid fibroblast cell materials, lack substantial moisture. In particular embodiments, the non-live fibroblasts and/or fibroblast-derived materials are dehydrated or lyophilized. In specific embodiments, the fibroblast-cell and fibroblast spheroid derived materials comprise exosomes, lysates, membranes, apoptotic bodies, growth factors, cytokines, chemokines, or a combination thereof, at least some of which are sourced from fibroblasts.

Embodiments of the disclosure may comprise one or more compositions comprising a wound coverage substrate, said composition comprising dehydrated or lyophilized non-live fibroblasts, and/or fibroblast-derived materials from single fibroblast cell culture, or spheroid fibroblast cell and/or dehydrated or lyophilized fibroblast-derived materials. In specific embodiments, the wound coverage substrate comprises a bandage, a powder, a film, an ointment, a cream, a table, a capsule, or a combination thereof. In some embodiments, the fibroblast-derived materials comprise exosomes, lysates, membranes, apoptotic bodies, growth factors, cytokines, chemokines, or a combination thereof. The source of the exosomes may comprise fibroblast cultures comprising bioactive molecules that promote wound healing. The source of the lysates may comprise fibroblast cultures comprising one or a mixture of proteins, growth factors, and/or other bioactive molecules. The source of the fibroblast fragments may comprise controlled mechanical disruption of fibroblast cultures.

In certain embodiments, the composition is comprised in a container with a pharmaceutically acceptable excipient. The composition may be comprised in a container with a pharmaceutically acceptable excipient and optionally a dispenser of the composition. Examples of dispensers include an aerosolizer, a spray gun, a pump spray dispenser, or a combination thereof. The composition may further comprise one or more immunogenetic biopolymers.

In various embodiments, there are methods of producing or modulating a composition as encompassed herein, comprising the step of exposing the composition or a part thereof to at least one source of moisture. In some cases, the source of the moisture is from the body of an individual, such as from a wound, such as blood, pus, wound drainage, or a combination thereof. In specific embodiments, the source of the moisture is an optionally sterilized aqueous liquid. In various embodiments, the source of the moisture is an aerosolizer, a spray gun, a pump spray dispenser, or a combination thereof. Some methods may include the step of the lyophilized or dehydrated composition being added to a bandage, a powder, a film, an ointment, a cream, a tablet, a capsule, or a combination thereof.

In an embodiment, there is a method of treating one or more wounds of an individual, comprising the step of applying an effective amount of any composition encompassed herein to the wound. In certain embodiments the wound is chronic (e.g., present at least in part for greater than about 4-6 weeks), although it may be acute in some cases. The wound may be in an individual that has diabetes, cancer, and/or is immunologically compromised. The wound may be of any kind, including a cut, abrasion, bedsore, burn, or the individual has more than one of these. Some embodiments of the disclosure relate to a composition including a wound coverage substrate. The composition can include dehydrated or lyophilized non-live fibroblasts and/or dehydrated or lyophilized fibroblast-derived materials.

In some embodiments, the wound coverage substrate includes a bandage, a powder, a film, an ointment, a cream, a table, a capsule, or a combination thereof.

In some embodiments, the composition includes dehydrated non-live fibroblast cells and/or dehydrated fibroblast-derived materials.

In some embodiments, the composition includes lyophilized non-live fibroblast cells and/or lyophilized fibroblast-derived materials.

In some embodiments, the fibroblast-derived materials include exosomes, lysates, membranes, apoptotic bodies, growth factors, cytokines, chemokines, fragments of fibroblasts or a combination thereof.

In some embodiments, the source of the exosomes includes fibroblast cultures including bioactive molecules that promote wound healing.

In some embodiments, the source of the lysates includes fibroblast cultures comprising one or a mixture of proteins, growth factors, and/or other bioactive molecules.

In some embodiments, the source of the of the fibroblast fragments includes controlled mechanical disruption of fibroblast cultures.

Some embodiments of the disclosure relate to any of the compositions disclosed herein, wherein the composition is included in a container with a pharmaceutically acceptable excipient.

In some embodiments, the composition is included in a container with a pharmaceutically acceptable excipient and optionally a dispenser of the composition.

In some embodiments, the dispenser includes an acrosolizer, a spray gun, a pump spray dispenser, or a combination thereof.

In some embodiments, the composition further includes one or more immunogenetic biopolymers.

In some embodiments, the fibroblasts of the methods or compositions described herein are selected fibroblasts or the fibroblast-derived materials are derived from selected fibroblasts. The selected fibroblasts can be selected for expression or secretion of HGF, KGF, FGF, VEGF, IL-6, IL-12, TNF-α, IFN-γ, CXCL1, CX3CL1, CCL2, angiopoetinangiopoietin 1, and/or thrombospondin. In some embodiments, the selected fibroblasts are selected for expression of CD90, CD,26, and/or S1004A.

Some embodiments of the disclosure relate to a method of producing or modulating any composition described herein. The method can include the step of exposing the composition or a part thereof to at least one source of moisture. In some embodiments, the source of the moisture is a wound. In some embodiments, the source of the moisture is an optionally sterilized aqueous liquid. In some embodiments, the source of the moisture is an aerosolizer, a spray gun, a pump spray dispenser, or a combination thereof.

In some embodiments, the method further includes the step of the lyophilized or dehydrated composition being added to a bandage, a powder, a film, an ointment, a cream, a table, a capsule, or a combination thereof.

Some embodiments of the disclosure relate to a method of treating one or more wounds of an individual. In some embodiment, the method includes the step of applying an effective amount of any of the compositions described herein. In some embodiments, the wound is chronic.

In some embodiments, the individual has diabetes, cancer, or is immunologically compromised. In some embodiments, the wound is a cut, abrasion, bedsore, burn, or the individual has more than one of these.

Some embodiments of the disclosure relate to a kit comprising any composition described herein. The kit can include one or more elements in individual or bulk packaging.

Some embodiments of the disclosure relate to a kit comprising means to carry out the method of any one of claims 15-23.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DETAILED DESCRIPTION

### I. Examples of Definitions

The use of the word “a” or “an” when used in conjunction with the term “comprising” may mean “one,” but it is also consistent with the meaning of “one or more,” “at least one,” and “one or more than one.”

The phrase “and/or” means “and” or “or.” To illustrate, A, B, and/or C includes: A alone, B alone, C alone, a combination of A and B, a combination of A and C, a combination of B and C, or a combination of A, B, and C. In other words, “and/or” operates as an inclusive or.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Throughout this specification, unless the context requires otherwise, the words “comprise,” “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the term “activated immune cells” refers to immune cells treated with one or more stimuli capable of inducing one or more alterations in the cell: metabolic, immunological, epigenetic, growth factor secreting, surface marker expression, and production and excretion of microvesicles.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration, oral ingestion, transdermal patch, topical, inhalation, suppository etc.

As used herein, “allogeneic” refers to tissues or cells from another body that in a natural setting are immunologically incompatible or capable of being immunologically incompatible, although from one or more individuals of the same species.

As used herein, “autologous” refers to tissues or cells that are derived or transferred from the same individual's body (i.e., autologous blood donation; an autologous bone marrow transplant).

As used herein, “agent” may refer to nucleic acids, cytokines, chemokines, transcription factors, epigenetics factors, growth factors, hormones, or a combination thereof.

As used herein, “xenogeneic” refers to tissues or cells from a species different from the patient.

“Cell culture” is an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO₂. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum.

The term “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adults and juveniles (i.e., children) and infants. It is not intended that the term “individual” connotes a need for medical treatment, therefore, an individual may voluntarily or involuntarily be part of experimentation whether clinical or in support of basic science studies. The term “subject” or “individual” may be used interchangeably and refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “transplantation” refers to the process of taking living tissue or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

As used herein, the term “effective amount” refers to a quantity sufficient to achieve a desired therapeutic effect, e.g., an amount which results in the decrease in a wound described herein or one or more signs or symptoms associated with a wound described herein. In the context of therapeutic applications, the dressing administered to the subject will vary depending on the composition, the degree, type, and severity of the wound and on the characteristics of the individual.

The term “fibroblast-derived material” as used herein refers to fibroblast cell fragments, conditioned media, exosomes secreted from fibroblasts, and/or fibroblast lysate and includes fibroblast-like cells; and/or extracellular vesicles including exosomes, microvesicles, apoptotic bodies or any other fragments or biologic components of fibroblast cells. For example, fibroblast cell-derived material is any material produced by or isolated from a fibroblast. For example, fibroblast spheroid-derived material is any material produced by or isolated from a fibroblast from a fibroblast spheroid. The term “spheroid” as used herein refers to an aggregate or assembly of cells cultured to allow 3D growth, as opposed to growth as a monolayer or a single cell.

It is contemplated that any embodiment discussed in this specification can be implemented with respect to any method or composition of the invention, and vice versa. Furthermore, compositions of the invention can be used to achieve methods of the invention.

### II. General Embodiments

The present disclosure introduces a novel wound healing system that in particular embodiments utilizes a bandage coated at least on one side with a specialized composition of non-live fibroblasts, and/or fibroblast-derived materials from single fibroblast cell culture, or spheroid fibroblast cell and/or fibroblast-derived materials such as, but not limited to, exosomes, lysates, membranes, apoptotic bodies, growth factors, cytokines and/or chemokines. Various embodiments of this system significantly enhance the wound healing process by delivering one or more therapeutic factors and/or cellular components useful for wound healing directly to the wound site, promoting faster tissue regeneration and improved wound closure. In certain embodiments, one could use aerosol, a pump spray, or a spray gun to apply the mixture to the wound. In such cases, the bandage may or may not also comprise the composition. In some embodiments, one could utilize an ointment dispenser to apply the mixture directly on or near the wound, or on a wound coverage substrate. In another embodiment, the fibroblast-derived materials such as, exosomes, lysates, membranes, apoptotic bodies, growth factors, cytokines and/or chemokines useful for wound healing could be dehydrated into a film comprising a non-immunogenetic biopolymer attached to the top surface of the bandage [3-6].

Embodiments of the wound healing system comprise a wound coverage substrate coated with non-live fibroblasts, and/or fibroblast-derived materials from single fibroblast cell culture, or spheroid fibroblast cell and/or a mixture of fibroblast-derived materials, such as exosomes, lysates, membranes, apoptotic bodies, growth factors, cytokines, chemokines, and/or any other component conducive to wound healing. Fibroblasts may be cultured with the appropriate cytokines, chemokines, and/or growth factors useful to prime the cells for the wound healing process, followed by harvesting to isolate the cells, and purifying the fibroblast-derived materials. The fibroblast-derived materials may then be lyophilized, such as for use in aerosol, pump spray, or spray gun, or as an additive in ointment, or they may be dehydrated into a film containing a non-immunogenetic biopolymer attached to the top surface of the bandage [7], in particular embodiments. The non-live fibroblasts, and/or fibroblast-derived materials from single fibroblast cell culture, or spheroid fibroblast cell and/or a mixture of fibroblast-derived materials, such as exosomes, lysates, membranes, apoptotic bodies, and growth factors, cytokines, and chemokines, and/or any other component useful for wound healing can be dehydrated or lyophilized.

Embodiments of the disclosure encompass a wound healing system, comprising a wound coverage substrate coated with a composition comprising non-live fibroblasts, and/or fibroblast-derived materials from single fibroblast cell culture, or spheroid fibroblast cell materials such as, exosomes, lysates, membranes, apoptotic bodies, growth factors, cytokines chemokines and/or any other component useful for wound healing or a combination of these. In some embodiments, the source of the fibroblasts or fibroblast-derived materials may be autologous, allogeneic, syngeneic, or xenogeneic with respect to an individual.

In certain embodiments, when fibroblast-derived exosomes are utilized, the exosomes may be isolated from fibroblast cultures and may comprise bioactive molecules that promote wound healing. In certain embodiments, when fibroblast-derived lysates are utilized, they may be obtained from fibroblasts and may comprise a mixture of proteins, growth factors, and other bioactive molecules. In certain embodiments, fibroblast fragments are generated through controlled mechanical disruption of fibroblast cultures and are then utilized.

In particular embodiments, the composition may be a mixture that is dehydrated to improve longevity of the composition. In certain embodiments, the composition may be a mixture that is lyophilized to improve longevity of the composition. In some embodiments, the composition can have a shelf life of about 1, 6, 12, 24, 36, 48, or 60 months or more. In certain examples, the composition is lyophilized for use in aerosol, spray gun, or pump spray. In specific embodiments, the mixture is lyophilized and added as an additive to an ointment mix for application to the wound. In specific embodiments, the mixture is mixed with a non-immunogenetic biopolymer and lyophilized into a substrate (such as a film) for inclusion into the surface of the bandage non-adhesive pad which will be in direct contact with the wound.

In certain embodiments, the composition is rehydrated for use. In specific embodiments, the composition is rehydrated using the moisture from the wound. In specific embodiments, the composition is rehydrated using externally-provided means. In some cases, the externally-provided means are mechanized means.

Embodiments include a wound healing system comprising a wound coverage substrate coated with a composition comprising fibroblast or fibroblast-spheroid-derived materials such as exosomes, lysates, membranes, apoptotic bodies, growth factors, cytokines, chemokines and/or any other component useful for wound healing or a combination of these delivered in a kit form.

Embodiments include any of the methods described herein for making the composition or for using the composition. Other embodiments include the composition, or the cell population used in the composition.

### III. Detailed Embodiments

### Fibroblasts

Fibroblasts used in the methods or composition can be selected based on certain characteristics. These characteristics may include secretion of one or more the following factors such as HGF, KGF, FGF, VEGF, IL-6, IL-12, TNF-α, IFN-γ, CXCL1, CX3CL1, CCL2, angiopoietin 1, thrombospondin, and/or the like. The fibroblast cells and fibroblast-derived material can be characterized by the absence or presence of one or more cell surface markers such as CD90, CD26, S100A4, HLA-ABC, HLA-DR, CD34, CD45, CD142, and/or the like.

The non-live fibroblasts and/or fibroblast-derived materials used can lack substantial moisture. For example, the fibroblasts and or fibroblast-derived materials can comprise less than about 50%, 45%, 40%, 35%, 30%, 25%, 20%, 15%, 10%, 5%, 2.5%, 2%, 1%, 0.5%, 0.1% or less water, by weight. In particular embodiments, the non-live fibroblasts and/or fibroblast-derived materials are dehydrated and/or lyophilized.

Culturing of fibroblasts

Fibroblasts used in the compositions may be cultured with the appropriate cytokines, and/or chemokines, growth factors, and/or chemicals (such as nicotinic acid) useful to prime the fibroblast cells, spheroids, or their derived materials for the wound healing process and/or to begin to express and/or secrete of one or more factors such as HGF, KGF, FGF, VEGF, IL-6, IL-12, TNF-α, IFN-γ, CXCL1, CX3CL1, CCL2, angiopoietin 1, thrombospondin, and/or the like or expression on the surface of cells or fibroblast derived material markers such as CD90, CD,26, S1004A, and/or the like, followed by harvesting to isolate the cells, and purifying the fibroblast-derived materials. Fibroblast cells and spheroids are cultured in a manner to maintain the desired secretion and surface marker characteristics, and are checked using methods such as ELISA, Luminex, flow cytometry, and/or the like to make certain of the desired characteristics. Cells, spheroids, and cell and spheroid derived material are purified using commercially available methods.

Non-limiting examples of cytokines that can be used to culture with the fibroblasts include: IL-4, IL-6, IL-10, IL-13, TNF-α, IL-1, IL-6, TGFB, TGFB1, CRP, and/or the like. Non-limiting examples of chemokines that can be used to culture with the fibroblasts include: CCL2, CCL3, CCL5, CCL7, CXCL1, CXCL2, CXCL3, CXCL4, CXCL5, CXCL6, CXCL7, CXCL8, CXCL9, CXCL10, CXCL11, CXLC12, and/or the like. Non-limiting examples of growth factors that can be used to culture with the fibroblasts include: KGF, EGF, HGF, PDGF, VEGF, TGF, TGF-B, FGF, and/or the like.

### Dehydrating of Fibroblasts

In particular embodiments, the non-live fibroblasts and/or fibroblast-derived materials are dehydrated. The process of dehydration can involve air drying, air drying in the presence of a vacuum, and/or air drying or vacuum airdrying in the presence of an optimized concentration of trehalose (or the like) which can range from 10-300 mM (e.g., about 10, 20, 50, 100, 150, 200, 250, 300 mM). Any dehydration method known in the art can be used.

### Lyophilizing of Fibroblasts

In particular embodiments, the non-live fibroblasts and/or fibroblast-derived materials are lyophilized. Any standard lyophilization method of cells and/or cell-derived materials utilized in the pharmaceutical industry can be used to lyophilize fibroblast cell, fibroblast spheroids, and/or fibroblast cell, fibroblast spheroid derived materials. In some embodiments, one or more or a combination of cytokines, chemokines, and/or growth factors, and/or cell culture media components can be lyophilized along with the fibroblast cells, fibroblast spheroids, and/or fibroblast cell-derived and/or fibroblast spheroid-derived material.

Non-limiting examples of cytokines, chemokines, and/or growth factors have been provided above.

### Rehydrating of Fibroblasts

In particular embodiments, the dehydrated or lyophilized non-live fibroblasts and/or fibroblast spheroids and/or fibroblast cell or fibroblast spheroid-derived materials are rehydrated for activation using saline, BPS, and or any reagent optimized for pH and components optimized for the rehydration of lyophilized non-live fibroblasts and/or fibroblast spheroids and/or fibroblast cell or fibroblast spheroid-derived materials.

### Compositions

Embodiments of the invention include compositions comprising dehydrated or lyophilized non-live fibroblasts and/or fibroblast spheroids and/or fibroblast cell-derived or fibroblast spheroid-derived materials. The fibroblast cell-derived or fibroblast spheroid-derived materials can be dehydrated or lyophilized.

Compositions can include dehydrated or lyophilized non-live fibroblasts; fibroblast cells or spheroids; and/or dehydrated or lyophilized fibroblast-derived and/or fibroblast spheroid-derived materials. The composition concentration of the material can range from 0.1 mg to 1000 mg of lyophilized non-live fibroblasts and/or fibroblast spheroids and/or fibroblast cell or fibroblast spheroid-derived materials per dose or bandage. For example, about 0.1 mg, 1, 10, 50, 100, 150, 200, 250, 300, 350, 400, 450, 500, 550, 600, 650, 700, 750, 800, 850, 900, 950, or 1000 mg or more lyophilized non-live fibroblasts and/or fibroblast spheroids and/or fibroblast cell or fibroblast spheroid-derived materials per dose or per wound coverage substrate. The composition can include fibroblasts as the only cell type in the composition. For example, in some embodiments, the composition does not include stem cells, such as mesenchymal stem cells, or blood cells, or the like.

In some embodiments, the composition further includes one or more of a combination of proteins or growth factors such as, but not limited to, HGF, KGF, FGF, VEGF, IL-6, IL-12, TNF-α, IFN-γ, CXCL1, CX3CL1, CCL2, angiopoietin 1, thrombospondin, and/or the like. Such proteins or growth factors can be in a concentration of about 0.001 mM to 1000 mM. For example, the protein or growth factor can be in a concentration of about 0.001, 0.01, 0.1, 1, 10, 100, 500, 750, 1000 mM or more.

In some embodiments, of the composition can further include bioactive molecules such as adjuvants including one of, for example immunogenetic biopolymers such as chemical product-based, viral product-based, bacterial product-based adjuvants, or a mixture thereof. These can consist of one or more steroid creams, roflumilast, moisturizers, Salicylic acid, Coal tar, Vitamin D-based cream or ointment, Retinoid creams, Calcineurin inhibitors, antibiotic, Anthralin, or any combination thereof at a concentration of 0.001 mM to 1000 mM. Such bioactive molecules can be in a concentration of about 0.001 mM to 1000 mM. For example, the protein or growth factor can be in a concentration of about 0.001, 0.01, 0.1, 1, 10, 100, 500, 750, 1000 mM or more.

### Wound Coverage Substrate

The composition can be applied to a wound coverage substrate. The wound coverage substrate can be any substrate that covers a wound, such as a bandage substrate. These substrates can include adhesive and non-adhesive bandages of varying size, gauzes, non-adherent dressings, hydrocolloidal dressings, foam dressings, alginate dressings, transparent film dressings, hydrogel dressings, collagen and other biopolymer based dressings, composite dressings, negative pressure wound therapy (NPWT) dressings, and a combination thereof.

The composition can be directly embedded into one or more substrate.

The composition can self-adhere to one or more substrate.

### Kits

The present invention provides various kits comprising means for any of the methods described in this disclosure. The kit can include any of the compositions described herein. The kit can include one or more elements in individual or bulk packaging.

### Methods

Methods for treating one or more wounds of an individual are provided. The method can include applying an effective amount of any of the compositions disclosed herein. Upon application, there can be an increase in one or more of hemostasis, immune cell recruitment, cell migration, cell proliferation, tissue remodeling, or any other wound healing effect in the wound. Any of these conditions can be increased by 1%, 5%, 10%, 20%, 25%, 50%, 75%, 100%, 150%, 200%, 300%, or more compared to an untreated wound or a wound treated with a wound cover substrate without a composition provided herein. In some embodiments, upon application of the composition, the wound or symptom associated with the wound is treated by inhibiting, relieving, or slowing progression of a symptom of the wound. The progression of the wound or symptom associated with the wound can be inhibited, relieved, or slowed by 1%, 5%, 10%, 20%, 25%, 50%, 75%, 100%, 150%, 200%, 300%, or more compared to an untreated wound or a wound treated with a wound cover substrate without a composition provided herein. Progression can be determined by measuring by wound (e.g., surface area, length, width, depth, etc.) or using a wound imaging device or the like. Symptoms can include, but are not limited to, scaring, bleeding, inflammation, pain, redness, and/or the like.

The components within the compositions contributing to the wound healing effect can act synergistically.

### EXAMPLES

The following examples are included to demonstrate certain embodiments of the disclosure. It should be appreciated by those of skill in the art that the techniques disclosed in the examples which follow represent techniques discovered to function well in the practice of the invention, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the embodiments of the disclosure.

### Example 1

### Bandages with Dehydrated or Lyophilized Fibroblasts/Derivatives Accelerate Wound Healing

Bandages are prepared using the disclosed methods and tested on mice. Uniform wounds are created in each of these mice and are treated with bandages of the disclosed methods and compared to a control group. The length, width, and depth of wounds are measured every other day until the wounds healed, and the mice are euthanized for further analysis. Treatment effectiveness is assessed by healing time.

The experiment and the analysis of the captured data confirm that mice treated with bandages prepared by the disclosed methods show an accelerated wound healing rate compared to the control group.

### Example 2

### Bandages with Dehydrated Fibroblasts Compared to Bandages with Non-Dehydrated Fibroblasts

Bandages are prepared using the disclosed methods and tested on mice. Uniform wounds are created in each of these mice and are treated with bandages of the disclosed methods (dehydrated fibroblasts) or bandages with non-dehydrated fibroblasts. The length, width, and depth of these wounds are measured every other day until the wounds healed, and the mice are euthanized for further analysis. Treatment effectiveness is assessed by healing time

The experiment and the analysis of the captured data show that mice treated with bandages prepared by the disclosed methods show an accelerated wound healing rate compared to mice treated with non-dehydrated fibroblasts.

All of the methods disclosed and claimed herein can be made and executed without undue experimentation in light of the present disclosure. While the compositions and methods of this invention have been described in terms of particular embodiments, it will be apparent to those of skill in the art that variations may be applied to the methods and in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Although the present disclosure and its advantages have been described in detail, it should be understood that various changes, substitutions, and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

The following references, to the extent that they provide exemplary procedural or other details supplementary to those set forth herein, are specifically incorporated herein by reference.
1. Cialdai, F., C. Risaliti, and M. Monici, Role of fibroblasts in wound healing and tissue remodeling on Earth and in space. Front Bioeng Biotechnol, 2022. 10: p. 958381.
2. Junker, J.P., R.A. Kamel, E.J. Caterson, and E. Eriksson, Clinical Impact Upon Wound Healing and Inflammation in Moist, Wet, and Dry Environments. Adv Wound Care (New Rochelle), 2013. 2(7): p. 348-356.
3. Pardeshi, S.R., et al., Progress on Thin Film Freezing Technology for Dry Powder Inhalation Formulations. Pharmaceutics, 2022. 14(12).
4. Gabr, S.A. and A.H. Alghadir, Evaluation of the Biological Effects of Lyophilized Hydrophilic Extract of Rhus coriaria on Myeloperoxidase (MPO) Activity, Wound Healing, and Microbial Infections of Skin Wound Tissues. Evid Based Complement Alternat Med, 2019. 2019: p. 5861537.
5. Schmiedova, I., et al., Case Report: Freeze-Dried Human Amniotic Membrane Allograft for the Treatment of Chronic Wounds: Results of a Multicentre Observational Study. Front Bioeng Biotechnol, 2021. 9: p. 649446.
6. Sum, R., et al., Wound-healing properties of trehalose-stabilized freeze-dried outdated platelets. Transfusion, 2007. 47(4): p. 672-9.
7. Wong, A.Y.W., et al., Topical Biological Agents as Adjuncts to Improve Wound Healing in Chronic Diabetic Wounds: A Systematic Review of Clinical Evidence and Future Directions. Cureus, 2022. 14(7): p. e27180.

1. A composition comprising a wound coverage substrate, said composition comprising dehydrated or lyophilized non-live fibroblasts and/or dehydrated or lyophilized fibroblast-derived materials.

2. The composition of claim 1, wherein the wound coverage substrate comprises a bandage, a powder, a film, an ointment, a cream, a table, a capsule, or a combination thereof.

3. The composition of claim 1, wherein the composition comprises dehydrated non-live fibroblast cells and/or dehydrated fibroblast-derived materials.

4. (Currently Amended The composition of claims 1, wherein the composition comprises lyophilized non-live fibroblast cells and/or lyophilized fibroblast-derived materials.

5. The composition of claim 1, wherein the fibroblast-derived materials comprise exosomes, lysates, membranes, apoptotic bodies, growth factors, cytokines, chemokines, fragments of fibroblasts or a combination thereof.

6. The composition of claim 5, wherein the exosomes are derived from a source, wherein the source of the exosomes comprises fibroblast cultures comprising bioactive molecules that promote wound healing.

7. The composition of claim 5, wherein lysates are derived from a source, wherein the source of the lysates comprises fibroblast cultures comprising one or a mixture of proteins, growth factors, and/or other bioactive molecules.

8. The composition of claim 5, wherein the fibroblast fragments are derived from a source, wherein the source of the fibroblast fragments comprises controlled mechanical disruption of fibroblast cultures.

9. The composition of claim 1, wherein the composition is comprised in a container with a pharmaceutically acceptable excipient.

10. The composition of claim 1, wherein the composition is comprised in a container with a pharmaceutically acceptable excipient and optionally a dispenser of the composition.

11. The composition of claim 10, wherein the dispenser comprises an aerosolizer, a spray gun, a pump spray dispenser, or a combination thereof.

12. The composition of claim 1, wherein the composition further comprises one or more immunogenetic biopolymers.

13. The composition of claim 1 wherein the fibroblasts are selected fibroblasts or the fibroblast-derived materials are derived from selected fibroblasts, wherein the selected fibroblasts are selected for expression or secretion of HGF, KGF, FGF, VEGF, IL-6, IL-12, TNF-α, IFN-γ, CXCL1, CX3CL1, CCL2, angiopoietin 1, and/or thrombospondin.

14. The composition of claim 1 wherein the fibroblasts are selected fibroblasts, or the fibroblast-derived materials are derived from selected fibroblasts, wherein the selected fibroblasts are selected for expression of CD90, CD, 26, and/or S1004A.

15. A method of producing or modulating a composition of claim 1, comprising the step of exposing the composition or a part thereof to at least one source of moisture.

16. The method of claim 15, wherein the source of the moisture is a wound.

17. The method of claim 15, wherein the source of the moisture is an optionally sterilized aqueous liquid.

18. The method of claim 15, wherein the source of the moisture is an aerosolizer, a spray gun, a pump spray dispenser, or a combination thereof.

19. The method of claim 15, further comprising the step of the lyophilized or dehydrated composition being added to a bandage, a powder, a film, an ointment, a cream, a table, a capsule, or a combination thereof.

20. A method of treating one or more wounds of an individual, comprising the step of applying an effective amount of the composition of claim 1 to the wound.

21. The method of claim 20, wherein the wound is chronic.

22. The method of claim 20, wherein the individual has diabetes, cancer, or is immunologically compromised.

23. The method of claim 20, wherein the wound is a cut, abrasion, bedsore, burn, or the individual has more than one of these.

24. A kit comprising the composition of claim 1 comprising one or more elements in individual or bulk packaging.

25. A kit comprising means to carry out the method of claim 15.
